(19) United States
(12) Patent Application Publication
(10) Pub. No.: US 2008/0160050 A1
(43) Pub. Date: Jul. 3, 2008

(54) DENDRITIC CELL TUMOR INJECTION THERAPY AND RELATED VACCINE

(76) Inventor: Kenichiro Hasumi, Suginami-ku (JP)

Correspondence Address: ECKERT SEAMANS CHERIN & MELLOTT, 600 GRANT STREET, 44TH FLOOR, PITTSBURGH, PA, 15219, US

(21) Appl. No.: 11/931,578

(22) Filed: Oct. 31, 2007

Related U.S. Application Data
(60) Provisional application No. 60/855,906, filed on Oct. 31, 2006.

(51) Int. Cl. A61K 39/00; A61K 35/12

(52) U.S. Cl. 424/277.1; 424/93.7

(57) ABSTRACT

The present invention provides effective, safe, comprehensive combinatorial treatment methods that are safe and effective for reducing the size of tumor cells in tumor tissue of a patient. The methods include reducing the size of tumor cells in tumor tissue of a patient by collecting monocyte cells from a patient; culturing the monocyte cells with one or a plurality of factors to form immature dendritic cells from the monocyte cells; introducing the immature dendritic cells and an adjuvant into the tumor tissue of the patient; and introducing activated T cells into the tumor tissue of the patient. The methods further comprise pretreating a patient with local chemotherapy, systemic chemotherapy, tumor irradiation or systemic chemotherapy in combination with local tumor irradiation prior to introducing the immature dendritic cells, adjuvant and activated T cells into the tumor tissue of the patient. Also provided is a cancer vaccine for reducing the size of a tumor in a patient, comprised of immature dendritic cells derived from monocytes collected from the patient, an adjuvant, tumor antigens from the patient, and activated T cells.

### CROSS REFERENCE TO RELATED APPLICATIONS

The present application claims priority to U.S. Provisional Application No. 60/855,905, filed Oct. 31, 2006, which is incorporated herein by reference.

### BACKGROUND OF THE INVENTION

1. Field of the Invention

The present invention relates to treatment methods for cancer patients and, more particularly, to combinatorial treatment methods employing conventional cancer therapies with cancer immunotherapy.

2. Description of the Prior Art

Dendritic cells (DCs) are the sentinel antigen presenting cells of the immune system (Banchereau, J. et al., Ann NY Acad Sci, 987:180-7, 2003). These cells have the capacity to acquire antigenic material from their environment and to subsequently initiate vigorous immune responses. Recognizing this potential, DCs have been used as a platform to deliver candidate vaccines for cancer immunotherapy (den Brok, M. B. et al., Expert Rev Vaccines, 4:699-710, 2005). In the majority of these studies, immature DCs are developed in vitro from monocytes or stem cell precursors, armed with the candidate vaccines in the form of tumor cell lysates, proteins or HLA class I allele specific peptides. The DCs are matured with selected cytokines and administered by intravenous or parenteral routes.

As an alternative approach, injection of immature DCs with adjuvant directly into a patient's tumor tissue theoretically allows these cells to acquire a variety of products of the tumor cell at the injection site capable of enhancing the generation of an immune response to a broad array of antigenic determinants (Chi, K. H. et al., J Immunother, 28:129-35, 2005).

Combining intratumoral injection of immature DCs with conventional treatments, such as radiotherapy and chemotherapy, has been suggested to augment immune responses and improve clinical outcome (Hoffmann T. K. et al., Cancer Res, 60:3542-9, 2000). Such treatments can induce apoptosis and create a milieu which enhances the ability of DCs to take up tumor antigens, mature and migrate to regional lymph nodes where they efficiently present the tumor antigens to T cells (Pierre, P. et al., Nature, 388:787-92, 1997). Administration of chemotherapy to control immunosuppressive regulatory T cells or injection with other cellular products, such as activated T (AT) cells, also has been suggested to help enhance immune responsiveness (Berd, D. et al., Cancer Res, 44:5439-43, 1984). Cyclophosphamide, for example, has been recognized to not only exert immunostimulatory effects, but also to decrease the number of regulatory T cells and enhance apoptosis (Berd, D. et al., Cancer Res, 44:5439-43, 1984). Infusions of autologous AT cells, which express CD4OL and are potent inducers of DC maturation, have been clinically shown to be well-tolerated. Infused cells are able to significantly expand in vivo and maintain a broad T cell spectrum (Berger C. et al., Blood, 101: 476-84, 2003). The administration of large numbers of AT cells, therefore, has the potential to increase the pool of T cells capable of reacting against tumors. Moreover, the release of cytokines, such as IFNγ by T cells, may further enhance antitumor effects.

There exists a need, however, to provide a more effective treatment protocol for patients with cancer.

### SUMMARY OF THE INVENTION

The present invention meets this need by providing comprehensive combinatorial treatment methods for reducing the size of tumor cells in tumor tissue of a patient.

In an aspect of the present invention, a method is provided for reducing the size of tumor cells in tumor tissue of a patient, comprising collecting monocyte cells from a patient; culturing the monocyte cells with one or a plurality of factors to form immature dendritic cells from the monocyte cells; introducing the immature dendritic cells and an adjuvant into the tumor tissue of the patient; and introducing activated T cells into the tumor tissue of the patient. The activated T cells may be introduced several days following introducing the immature dendritic cells and the adjuvant in the patient, in order to generate antigen-specific T cells.

Introduction of the immature dendritic cells, adjuvant and activated T cells may be effected by intratumoral injection.

The monocyte cells of the patient, i.e., peripheral blood mononuclear cells (PBMCs), may be cultured with factors such as, for example, GM-CSF and IL-4.

Activation of T cells may be effected by exposing T cells to anti-CD3 antibodies and an ionophore such as, for example, ionomycin.

Activated lymphocyte medium may be prepared by using CD3-CD28 beads.

Suitable adjuvants to be used in the methods of the present invention include, without limitation, activated lymphocyte medium (ALM), super lymphoid tissue extract, β-glucan or keyhole limpet hemocyanin.

In another aspect of the present invention, there is provided a method for reducing the size of tumor cells in tumor tissue of a patient, further comprising pretreating a patient with local chemotherapy prior to introducing the immature dendritic cells, adjuvant and activated T cells into the tumor tissue of the patient.

In another aspect of the present invention, there is provided a method for reducing the size of tumor cells in tumor tissue of a patient, further comprising pretreating a patient with systemic chemotherapy prior to introducing the immature dendritic cells, adjuvant and activated T cells into the tumor tissue of the patient.

In another aspect of the present invention, there is provided a method for reducing the size of tumor cells in tumor tissue of a patient, further comprising pretreating a patient with systemic chemotherapy and local tumor irradiation prior to introducing the immature dendritic cells, adjuvant and activated T cells into the tumor tissue of the patient.

In another aspect of the present invention, there is provided a cancer vaccine precursor which when introduced into tumor tissue combines with an antigen for the reduction of the size of a tumor in a patient, in which the cancer vaccine comprises immature dendritic cells derived from monocytes collected from the patient, an adjuvant, antigens from the tumor in the patient, and activated T cells.

It is an object of the present invention to provide a more safe, effective combinatorial treatment protocol for patients with cancer.

It is another object of the present invention to provide an effective, safe method for reducing the size of tumor cells in a patient using conventional therapy(s) plus intratumoral injection of immature dendritic cells, an adjuvant and activated T cells.

It is another object of the present invention to provide an effective, safe cancer vaccine for reducing the size of a tumor in a patient, in which the cancer vaccine comprises immature dendritic cells, an adjuvant and activated T cells.

### BRIEF DESCRIPTION OF THE DRAWINGS

A full understanding of the invention can be gained from the following description of the preferred embodiments when read in conjunction with the accompanying drawings in which:

FIG. 1 provides four treatment protocols in accordance with embodiments of the invention;

FIG. 2 shows PET CT imaging of recurrence of upper pharyngeal cancer in a 45 year old female (A) before, (B) about 3 weeks, (C) about 3.5 months, and (D) 9 months following AT cell treatment; and

FIG. 3 is a graph illustrating that CEA serum levels drop following treatment with dendritic cells and AT cells (Protocol IV).

### DESCRIPTION OF THE PREFERRED EMBODIMENTS

The present invention provides comprehensive combinatorial treatment methods that are safe and effective for reducing the size of tumor cells in tumor tissue of a patient.

In an embodiment of the present invention, there is provided a method for reducing the size of tumor cells in tumor tissue of a patient, comprising collecting monocyte cells from a patient; culturing the monocyte cells with one or a plurality of factors to form immature dendritic cells from the monocyte cells; introducing the immature dendritic cells and an adjuvant into the tumor tissue of the patient; and introducing activated T cells into the tumor tissue of the patient. The activated T cells may be introduced several days following introducing the immature dendritic cells and the adjuvant in the patient, in order to generate antigen-specific T cells.

Introduction of the immature dendritic cells, adjuvant and activated T cells may be effected by intratumoral injection.

The monocyte cells of the patient, i.e., peripheral blood mononuclear cells (PBMCs), may be cultured with factors such as, for example, GM-CSF and IL-4.

Activation of T cells may be effected by exposing T cells to anti-CD3 antibodies and an ionophore such as, for example, ionomycin.

Activated lymphocyte medium may be prepared by using CD3-CD28 beads.

Suitable adjuvants to be used in the methods of the present invention include, without limitation, activated lymphocyte medium, super lymphoid tissue extract, β-glucan or keyhole limpet hemocyanin.

In another embodiment of the present invention, there is provided a method for reducing the size of tumor cells in tumor tissue of a patient, further comprising pretreating a patient with local chemotherapy prior to introducing the immature dendritic cells, adjuvant and activated T cells into the tumor tissue of the patient.

In another embodiment of the present invention, there is provided a method for reducing the size of tumor cells in tumor tissue of a patient, further comprising pretreating a patient with systemic chemotherapy prior to introducing the immature dendritic cells, adjuvant and activated T cells into the tumor tissue of the patient.

In another embodiment of the present invention, there is provided a method for reducing the size of tumor cells in tumor tissue of a patient, further comprising pretreating a patient with systemic chemotherapy and local tumor irradiation prior to introducing the immature dendritic cells, adjuvant and activated T cells into the tumor tissue of the patient.

In another embodiment of the present invention, there is provided a cancer vaccine precursor which when introduced into tumor tissue combines with an antigen for the reduction of the size of a tumor in a patient, in which the cancer vaccine comprises immature dendritic cells derived from monocytes collected from the patient, an adjuvant, antigens from the tumor in the patient, and activated T cells.

As used herein, “patient” is meant to refer to mammals, including humans.

The present invention is more particularly described in the following non-limiting example, which is intended to be illustrative only, as numerous modifications and variations therein will be apparent to those skilled in the art.

### Example

### I. Introduction

A pilot safety trial was conducted to investigate whether combining conventional treatment with DC and AT cell therapy would be well-tolerated. Several protocols were developed, in which immature DCs and AT cells were prepared from peripheral blood and injected into metastatic lesions in patients with advanced stages of malignancies. Patients first were injected with autologous immature DCs and adjuvant on day 0. Once it was established that the immature DC injection did not induce adverse reactions, additional treatment modalities known to augment immune responses without substantial toxicity were incorporated into different treatment arms. Radiotherapy or chemotherapy was given prior to immature DC administration. Specific tumor areas were subjected to radiotherapy (20-50Gy) to elicit antigenicity resulting from fragmentation of tumor DNA. In some cases, chemotherapeutic agents, including cisplatin (CDDP), cyclophosphamide (Cytoxan), fluorouracil (5-FU), Docetaxel (DTX) or Adriamycin (ADM) used in conventional therapy for the specific malignancy, were given in order to induce apoptosis or reduce the presence of cells that might suppress the immune response. AT cell therapy was administered 3 days following immature DC and adjuvant treatment.

### II. Patients and Methods

1. Patient Treatment

Thirty-seven patients aged 41-83 years old were enrolled in the DC and AT cell therapy study protocols. All patients had relapsed after receiving and failing standard chemotherapy and/or radiation indicated for their cancer. The types of cancer and number of cases included in the study are listed in Table 1. Patients were placed in study protocols, summarized in Table 2.

TABLE 1
Clinical Cases Treated with DC-based Therapy

| Type of Cancer | Number of Cases (n = 37) |
|---|---|
| Breast | 5 |
| Colorectal | 10 |
| Endometrial uterine | 1 |
| Esophageal | 1 |
| Gastric | 10 |
| Lung | 2 |
| Malignant melanoma | 1 |
| Malignant mesothelioma | 1 |
| Nasopharyngeal | 2 |
| Ovarian | 1 |
| Primary unknown | 1 |
| Renal pelvis | 1 |
| Thyroid | 1 |

TABLE 2
Summary of Study Protocols DC intratumoral
injection with adjuvant plus:

| Protocol No. | | Chemotherapy | | | |
|---|---|---|---|---|---|
| (No. cases) | | Local tumor | Systemic | Radiation | AT cells |
| I | (8) | ~ | ~ | ~ | ~ |
| II | (10) | X | ~ | ~ | ~ |
| III | (14) | X | ~ | ~ | X |
| IV | (3) | ~ | X | ~ | X |
| V | (2) | ~ | X | X | X |

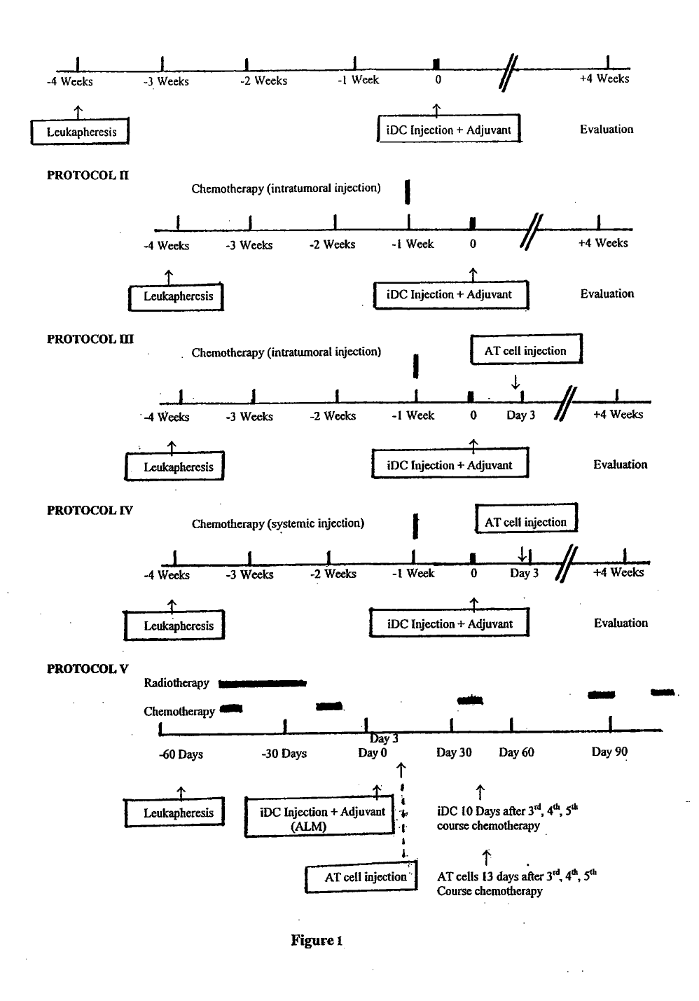

All patients received immature DCs (ranging from 4.9×10⁶ to 5.9×10⁹ per injection) and an adjuvant, either alone or combined with conventional chemotherapy, radiation and/or AT cells (4.1×10¹ to 7.5×10⁸). Prior to treatment, tumors were biopsied and blood tests performed. FIG. 1 illustrates the course of treatment in each of five protocols developed successively. All patients were given immature DCs and adjuvant on day 0. Patients enrolled in Protocol I received immature DCs and adjuvant. Protocol II patients were pretreated with local chemotherapy prior to receiving immature DCs and adjuvant. In other groups of patients, AT cells also were given at three days following immature DC and adjuvant injection with or without pre-treatment with chemotherapy or radiation. Chemotherapy was administered intratumorally (Protocol III) or systemically (Protocol IV) or combined with radiation (Protocol V). Patients were followed for evidence of local and/or systemic adverse reactions. Tissue biopsies and blood tests were conducted 3-4 weeks following therapy. Local or metastatic tumor regression was evaluated by PET-CT imaging. If tumor regression was observed, treatment was repeated.

2. Collection and Isolation of Peripheral Blood Mononuclear Cells (PBMCs)

Leukaphereses were performed on a COBE Spectra blood separator (Gambro KK, Tokyo, Japan) using the program for collection of mononuclear cells (MNCs) (version 7.1) with manual adjustments of the plasma pump. Anticoagulation was achieved with ACD-A (ratio of 12:1, Baxter, Deerfield, Ill.). The inlet rates were 40-60 ml/min with a collection rate of 1 ml/min and a separation factor of 700. MNCs were ficolled, cryopreserved at 1.5×10⁸ cells/ml/vial in 10% DMSO in AIM-V medium (Gibco, Invitrogen, Tokyo), and stored at −80° C. for 1-3 months. Prior to use in DC and AT cell culture, MNCs were thawed in a 3 V waterbath, washed twice in AIM-V medium, and counted.

3. Generation of Immature DCs (iDCs)

Thawed MNCs (approximately 6×10⁸) were resuspended in 20 ml AIM-V medium and distributed in 5 ml aliquots to 4-T-75 cm² polystyrene flasks each containing 1 Oral of AIM-V medium. Following a 2 h incubation at 37° C., non-adherent cells were removed by pipette, transferred to conical tubes and reserved for AT cell preparation (see below). Fifteen ml of DC growth medium (AIM-V medium supplemented with 800 IU/ml GM-CSF (CellGenix, Germany)+500 U/ml IL4 (BD Pharmingen) was added to each of the flasks containing the adherent cells. Flasks were incubated at 37° C., 5% CO₂. Growth media were refreshed on day 3 and DCs were harvested on day 7 by pipetting. Harvested cells were counted and resuspended in AIM-V freezing medium containing 20% autologous serum+10% DMSO, then cryopreserved in BICELL containers (Nihon Freezer Co., Tokyo, Japan), which allows for gradual freezing of cells similar to that of programmed freezing (1° C. per minute freezing rate). Cells were stored at −80° C. until patient injection (0.5-3 months).

4. Preparation of AT Cells

Non-adherent T cells (approximately 6-9×10⁸ cells) collected following monocyte adherence for DC generation were washed and resuspended in 20 ml AIM-V medium. Five ml of this cell suspension and 35 ml of AT cell medium was added to each of four T-225 cm² flasks coated with anti-CD3 antibody (Yamazaki, T. et al, Neurol Med Chir, Tokyo, 32:255-61, 1992). Flasks then were incubated for 7 days at 37° C., 5% CO₂. Three hours prior to harvesting, 1 ug/ml ionomycin (Sigma, USA) was added to the medium to stimulate T cells (Sato, T. et al., Cancer Immunol Immunother, 53:53-61, 2004). AT cell medium consisted of AIM-V medium supplemented with IL2 and autologous serum so that each flask contained a final level of 1000 IU/ml IL2 and 10% autologous serum. The anti-CD3 antibody-coating was prepared by adding 10 ml of 5 ug/ml anti-CD3 antibody (Orthoclone, OKT3 injection. Janssen Pharmaceutical, KK) in DPBS to flasks for 2 h at room temperature followed by washing the flasks 3 times with 15 ml DPBS prior to adding cells. Harvested cells were cryopreserved and stored at −80° C. prior to patient injection (0.5-3 months).

5. Sterility and Purity Testing of Cell Cultures

Seven days prior to harvesting cells, microbial testing was started by incubation of cultured cells on agar at 37° C. Only negatively tested cultures were used clinically. Cell purity was assessed by determination of endotoxin levels (<0.5 EU/ml) using a commercially available chromogenic endotoxin assay kit according to the manufacturer's instruction (Toxicolor system LS-50M, Seikagaku Corp., Tokyo, Japan). The concentration of endotoxin in each sample was measured with Limulus amoebocyte lysate (LAL). Briefly, 50 ul of each sample was transferred to an endotoxin-free 96-well plate (Toxipet LP; Seikagaku), and then 50 ul of the LAL reagent was added to each well, mixed and incubated at 37° C. for 30 min. To stop the reaction, 0.8 M acetic acid was added to each well, mixed and measured at 405 μm. Endotoxin levels were calculated against a reference endotoxin, E. coli 011 1:B4 LPS (Toxicolor system CSE; Seikagaku).

6. Characterization of Cell Cultures

A standard flow cytometry labeling protocol was used to determine cell surface marker expression (Parks, D. et al., Flow Cytometry and Fluorescence-Activated Cell Sorting; Raven Press Ltd, New York, 1989). DCs were characterized for surface marker expression after 7 days of culture and following thawing, prior to DC injection using fluorochrome-conjugated monoclonal antibodies against CD11c, CD14, CD40, CD80, CD83, CD86 and HLA-DR (BD Pharmingen). AT cells were evaluated for CD3, CD4, CD8, CD11c, CD14, CD19, CD25, CD45, CD56, CD154 (CD40L) and HLA-DR following culture. Minimally, 10,000 events were acquired on a BD FACscan (BD Biosciences) and data were analyzed using Cell Quest analysis software. Marker expression was presented as the mean level of fluorescence in patient cell cultures of the standard deviation.

7. Preparation of Cells for Injection

DCs and AT cell vials were thawed in a 37° C. waterbath 1 hour prior to planned injection. One ml of AIM-V medium was added to each thawed vial; vials were allowed to sit for 2 minutes and then transferred to 50 ml of media and centrifuged (300×g, 7 min) to remove DMSO. Cells were resuspended in fresh media, counted, and a sample removed for sterility testing by agar plating. Remaining cells were distributed to two labeled microtubes (500 ul each) and placed on ice for delivery to the clinic for injection into patient's metastatic lesions. Cells were injected based on CT scan information.

8. Preparation of Adjuvants

Different adjuvants were included in the protocols: Commercially available (1) super lymphoid tissue extract (S-LTE, 0.5 ml, Shukokai, Inc., Japan, Protocols I and II); (2) β-glucan (0.5 ml-1.0 ml at 50 ug/ml in saline, Wako, Japan, Protocols II and III); or (3) keyhole limpet hemocyanin (KLH), 1 mg, Immucothel, Protocol IV, prepared per manufacturer's recommendation; and (4) Activated Lymphocyte Medium (ALM); Protocols III, IV and V, prepared in our clinical laboratory from elutriated lymphocytes (Gambro BCT, Colorado, USA). To make ALM, lymphocytes were suspended at 1×10⁶ cells/ml in 50 ml XVIVO 10 medium (Cambrex, Walkersville, Md.) per T-75 cm² flask and CD3-CD28 T cell expander beads (Dynal. Norway) (Levine, B. L. et al., J Immunol, 159:5921-30, 1997) were added at 1 cell to 1 bead ratio and incubated at 37° C., 5% CO₂ for 2 days. The supernatants were harvested by centrifugation at 300×g for 7 min and stored at 4° C. for later use. All adjuvants were delivered intratumorally at the same time as delivery of DCs.

9. Chemotherapy

The type of chemotherapy was selected based on standard treatment for the particular cancer. Chemotherapy was administered intratumorally or systemically depending on the protocol. Twenty-one patients received Cyclophosphamide (CPM, 5-800 mg, Shinogi), five patients received Cisplatin (CDDP, 2.5-5.0 mg, Nihon Kayaku), one patient received Docetaxel (DTX, 55 mg, Aventis), and one patient received Doxorubicin (ADM, 20 mg, Kyowa Hakko). CDDP was administered systemically in combination with Fluorouracil (5-FU, 900 mg/day, Kyowa Hakko) in one patient with upper pharyngeal cancer.

10. Radiotherapy

Radiotherapy (20-50 Gy) was given to the two patients on Protocol V. One patient with metastatic upper pharyngeal cancer received 50 Gy and the other with recurrent metastatic esophageal cancer received 20 Gy. Radiotherapy was delivered to patients according to a defined protocol (Shimamura, H. et al., Eur Surg Res, 37:228-34, 2005). The radiation area of the tumor site was designated in advance and radiation was given at 2-2.5 Gy doses during a 3-4 week period. Radiotherapy was performed during the same period as chemotherapy. DCs were injected intratumorally seven days following the completion of radiotherapy and AT cells were injected intratumorally ten days following completion of radiotherapy or three days following DC injection in the applicable protocols.

11. Evaluation of Responses

Carcinoembryonic antigen (CEA) levels elevated in cancer, such as cancer of the colon, rectum, stomach, breast, lung or pancreas, were used to monitor patient responses to treatment and disease recurrence. Tumor size of the injected and irradiated sites, as well as metastatic sites, was evaluated prior to and 3-4 weeks following treatment using PET-CT imaging and Response Evaluation Criteria in Solid Tumors (RECIST). Baseline sums were assessed as the largest diameter in measurable lesions. The efficacy of treatment was assessed as complete response (CR, defined as complete disappearance of measurable lesions, with no new lesions developing and maintained for greater than 4 weeks), partial response (PR, defined as at least a 30% reduction in the size of injected tumors), stable disease (SD, defined as less than 30% reduction and less than 20% increase in size, with no new lesions developing) and progressive disease (PD, defined as 20% or greater increase of original measurements). Adverse effects were recorded using common WHO toxicity criteria.

12. Statistical Measures

Tumor size data pre- and post treatment to gauge changes were compared using the Student's t test and paired t test. Statistical significance was determined at p<0.05.

### III. Results

1. Characterization of DCs and AT Cells

Adherent PBMCs can be differentiated to DCs. Seven day cultured adherent cells from patients with a variety of cancers expressed costimulatory markers and low CD83 surface antigen, indicative of immature DCs (Table 3). Cell viability remained high at 85-95% following 7 days in culture, as well as following cryopreservation. Lymphocytes treated with anti-CD3 and ionomycin for activated T cell therapy expressed CD25 and CD40L (Table 3). The viability of the stimulated cells ranged from 75-90% prior to and following cryopreservation.

TABLE 3
Characterization of Cells Used in Treatment Protocols

| DC Cultures (n = 37) | AT Cell Cultures (n = 19) |
|---|---|
| % Expression | % Expression |
| Myeloid Markers | Lymphocyte Markers |

| | | | |
|---|---|---|---|
| CD11c⁺ | 96 ± 5 | CD3⁺ | 88 ± 11 |
| CD14⁺ | 74 ± 19 | CD4⁺ | 59 ± 15 |
| | | CD3⁺CD56⁻ (NKT) | 38 ± 26 |
| | | CD19⁺ | 7 ± 4 |

| | |
|---|---|
| Costimulatory Markers | Monocyte/NK Cell Markers |

| | | | |
|---|---|---|---|
| CD40⁺ | 88 ± 12 | CD14⁺ | 15 ± 12 |
| CD86⁺ | 92 ± 7 | CD56⁺ | 48 ± 29 |
| HLA-DR | 82 ± 15 | | |

| | |
|---|---|
| Maturation Marker | Activation Markers |

| | | | |
|---|---|---|---|
| CD83+ | 16 ± 16 | CD11c⁺ | 66 ± 22 |
| | | CD25⁺ | 87 ± 11 |
| | | CD154⁻ (CD40L) | 31 ± 15 |
| | | HLA-DR | 79 ± 12 |

2. Toxicity

Blood and urine tests conducted during and following treatment showed no changes in total protein, glutamate oxaloacetic transaminase (GOT), glutamic pyruvic transaminase (GPT), lactate dehydrogenase (LDH), alkaline phosphatase, total bilirubin, blood urea nitrogen (BUN), creatinine, total cholesterol, triglycerides, blood sugar, hematuria or proteinuria.

All treatments were generally well-tolerated with adverse reactions limited to a few patients in the groups receiving AT cell therapy (Table 4). No local or systemic adverse reactions were observed immediately after injection or during the follow-up period in patients receiving DCs+adjuvant on Protocols I and II. In patients receiving AT cell therapy, fever (38° C.) was observed in four patients on Protocol III just after injection and the two patients in Protocol V experienced injection site swelling, lymphadenopathy, and pain and fever.

TABLE 4
Adverse Reactions
Treatment

| | DCs + Adjuvant + | | DCs + Adjuvant + AT Cells + | | |
|---|---|---|---|---|---|
| | | Chemo | Chemo | Chemo | Chemo + |
| | Alone | (tumor) | (tumor) | (sys) | rad (sys) |
| | I | II | III | IV | V |
| Symptom | (n = 8) | (n = 10) | (n = 14) | (n = 3) | (n = 2) |
| Arthralgias | No | No | No | No | No |
| Blood Pressure | No | No | No | No | No |
| Changes | | | | | |
| Diaphoresis | No | No | No | No | No |
| Dyspnea | No | No | No | No | No |
| Erythema (at | No | No | No | No | No |
| injection site) | | | | | |
| Lymphadenop- | No | No | No | No | Yes (2/2)* |
| athy (regional | | | | | |
| nodes) | | | | | |
| Muscle Pain | No | No | No | No | No |
| Pain | | | | | |
| regional nodes | No | No | No | No | Yes (1/2)* |
| non-injected | — | — | — | — | Yes (2/2) |
| visible metas- | | | | | |
| tatic sites | | | | | |
| Palpitation | No | No | No | No | No |
| Pulsation | No | No | No | No | No |
| Changes | | | | | |
| Swelling | | | | | |
| injection site | No | No | No | No | Yes (2/2)* |
| non-injected | No | No | No | No | No |
| visible metas- | | | | | |
| tatic sites | | | | | |
| Transient Fever | — | — | Yes (4/14) | No | Yes (2/2)* |
| (38° C.) post | | | | | |
| AT injection | | | | | |

*Symptom appeared a few days following AT cell therapy and improved within 1 week; chemo = chemotherapy; rad = tumor irradiation; tumor = intratumoral injection; sys = systemic administration

3. Patient Response

Clinical efficacy was observed by comparing tumor size prior to therapy and 3-4 weeks following the first DC injection (Table 5).

TABLE 5
Effectiveness of Treatment

| | DCs + adjuvant + | | DCs + adjuvant + AT Cells + | | | |
|---|---|---|---|---|---|---|
| | | chemo | chemo | chemo | chemo + | |
| | — | tumor | tumor | sys | rad sys | |
| Response | I | II | III | IV | V | Total |
| CR | 0 | 0 | 0 | 0 | 1^{b} | 1 |
| PR | 2^{a} | 4^{a} | 0 | 0 | 0 | 6 |
| SD | 4 | 4 | 14 | 3 | 0 | 25 |
| PD | 2 | 2 | 0 | 0 | 1 | 5 |
| Total | 8 | 10 | 14 | 3 | 2 | |

CR = Complete Response; PR = Partial Response; SD = Stable Disease; PD = Progressive Disease; chemo tumor = tumor injection of chemotherapy; chemo sys = systemic administration of chemotherapy; rad = tumor irradiation;
^{a}Responses limited to DC-injected site;
^{b}PET-CT data showed improvement in both DC-injected and metastatic sites

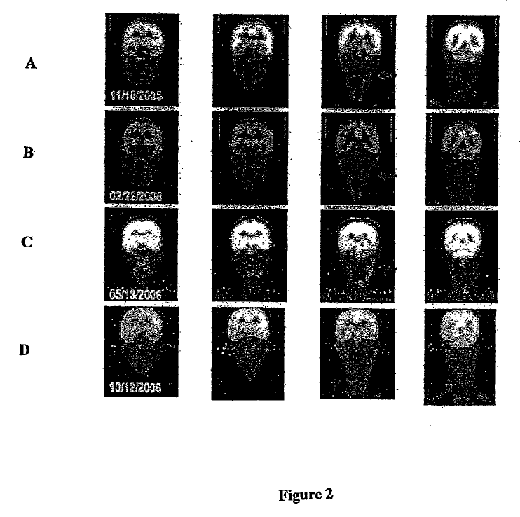

Seven of 37 patients (19%) responded to treatment with complete (one patient in Protocol V) or partial regression (six patients in Protocols I and II) in the tumor area which was observed for at least 3 months. Complete tumor regression was seen in a patient diagnosed with recurrence of upper pharyngeal cancer receiving DCs, AT cells and both chemotherapy and radiation (Protocol V, FIG. 2) with efficacy at both treated and metastatic sites.

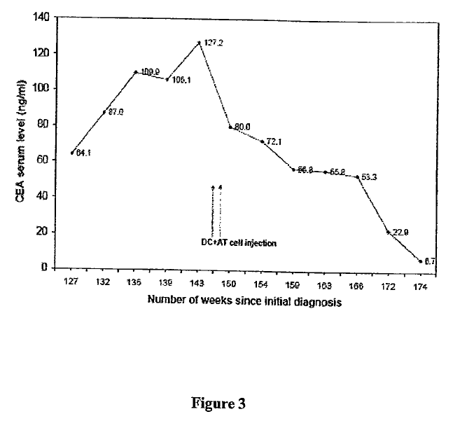

In 25 of 37 (67%) patients across all protocols, tumor size remained the same and did not change for at least 3 months. Serum tumor markers were followed in patients where indicated for a particular tumor and stabilized over this period of time. However, one patient with metastatic gastric adenocarcinoma showed a dramatic decline in CEA levels following injection of DCs and AT cells (FIG. 3). The patient was diagnosed approximately 3 years prior to enrollment in our protocol and had undergone multiple rounds of chemotherapy and a subtotal gastrectomy. Despite these standard treatment approaches, CEA levels continued to rise and there was an increase in size of the periaortic lymph nodes. The patient received 800 mg of cyclophosphamide prior to injection of DCs followed by AT cells. Serum CEA levels showed a steady decline reaching a level of 6.7 at the latest examination.

Progressive disease was observed in four patients receiving DCs alone or DCs with chemotherapy (Protocols I and II) and in one patient receiving DCs and AT cells with chemotherapy and radiation (Protocol V). In the latter, stable disease was observed for 1 month with progression at 3 months in untreated tumors.

### IV. Discussion

Anti-cancer treatments, including, tumor irradiation, chemotherapy and adoptive cellular therapy have shown effectiveness on their own. The primary purpose of this study was to determine the safety and efficacy of administering autologous immature DCs directly into tumors and then combining this approach with other treatment modalities such as direct injection of a metastatic lesion with activated T cells, pretreatment with local chemotherapy or radiation to the injection site or systemic chemotherapy, in a variety of advanced stage cancer patients. This study showed that combining the unique properties of the different treatment modalities within single protocols significantly augmented anti-tumor responses.

It is well-established that DCs serve as important antigen presenting cells for the induction of specific T cell immunity (Banchereau, J. et al., Nature 392: 245-52; 1998) and, as such, have formed the basis of a number of clinical trials in a variety of cancers. Some trials have employed ex vivo generated DCs loaded with tumor-specific antigens, which can result in induction of anti-tumor immunity when reinfused into the host (Hsu, F. J. et al., Nat. Med., 2:52-58, 1996). However, in cancers where tumor-associated antigens are not well defined, as in this study, the whole tumor has been used in the form of apoptotic cells. Apoptotic cells effectively cross-prime T cell responses and induce potent immunity.

Ionizing radiation and chemotherapy is used to induce apoptosis. Tumor irradiation typically produces inflammation due to release of intracellular contents into the surrounding environment accompanied by, but not limited to, activation of cytokines, prostaglandins and heat shock proteins. Local delivery of chemotherapy creates a similar environment at the tumor site. Such strategies produce a milieu that facilitates antigen acquisition, processing, and maturation, as well as stimulate migration of DCs to draining lymph nodes where they interact with a broad range of potential effector cells.

Combining conformal radiotherapy and chemotherapy with DC intratumoral injection improves immune responsiveness shown both in human and murine models and from a practical standpoint, direct injection can ensure adequate quantities of antigen for loading onto DCs.

Because the primary goal of this study was to assess the safety and efficacy of different treatments using immature DCs as the base immunization, a group of patients was first injected with DCs directly into untreated tumors (Protocol I). No adverse reactions were observed using this method of injection and some patients actually showed partial responses at the tumor site (25%, 2/8), some had stable disease (50%, 4/8), while disease progressed in some patients (25%, 2/8). No adverse reactions were observed in the next group of patients, when local tumor chemotherapy was added to enhance antigen uptake by DCs (Protocol II). The addition of local treatment actually seemed to slightly improve local partial tumor responses (40%, 4/10). Patients also developed stable (40%, 4/10) and progressive disease (20%, 2/10).

It has been suggested that tumor cells undergoing apoptosis may not induce immunity due to local secretion of immunosuppressive factors such as TGFO and IL10 and the lack of enough systemic functional DCs to reach the peritumoral region (Bodey, B. et al., Anticancer Res., 20: 2665-76, 2000). The tumor environment determines the state of maturation which can result in the induction of tolerance or immunity. Therefore, to complete DC activation at the injected site, other proinflammatory cytokines or immune-provoking substances may need to be combined with the immature DCs.

Combining DCs with other cell types, such as AT cells, therefore, is a promising therapy. DCs activate T cells and their interaction is important for the establishment of an effective immune response. Activated T cells could influence antigen presentation by DCs by a number of mechanisms. AT cells express surface molecules that physically interact with immature DCs and in addition produce cytokines and chemokines that influence the maturation and migration of DCs to regional lymph nodes where adaptive immune responses to acquired antigens are generated. As a consequence, a tumor specific immune response potentially is developed and long-term memory established.

There are different strategies in generating T cells for adoptive therapy which influence not only their antigen-specificity, tumor avidity and cellular phenotype, but also their behavior in vivo such as longevity, trafficking and anti-tumor efficacy. T cells have been expanded by a variety of methods including in vitro stimulation to elicit antigen-specific T cells from peripheral blood using cells engineered for antigen presentation (e.g., DCs pulsed with peptide or RNA transfected) or artificial antigen presenting cells. Harvesting infiltrating lymphocytes from tumor cells yield polyclonal T cell populations with broad reactivity to tumor. Re-infusing these cells after in vitro expansion has been used as an immunotherapeutic treatment modality. Another approach has been to activate and expand peripheral blood lymphocytes by triggering the T cell receptor and costimulatory molecules with antibodies and/or the use of cytokines to drive T cells.

In this study, AT cells were expanded from peripheral blood lymphocytes by anti-CD3-IL2 culture and ionomycin activation to upregulate CD40L. Cultured AT cells were injected intratumorally, several days following DC injection, with the idea that antigen-specific T cells could be generated. Patients receiving AT cell therapy were pretreated with: I) local chemotherapy (Protocol III), 2) systemic chemotherapy (Protocol IV), or 3) systemic chemotherapy and local tumor irradiation (Protocol V).

When local tumor irradiation was added in the third AT cell protocol (Protocol V), transient fever, lymphadenopathy, pain in metastatic sites, and swelling in the injection site following AT cell injection appeared in two patients. These symptoms improved within 1 week and remarkably, in one patient with pharyngeal cancer, complete tumor regression of both treated and untreated sites was observed. Beginning at 3 weeks following treatment, except for the injection site, there was regression in all areas including the pharynx, larynx and bilateral cervical lymph node and by the nine-month follow up, tumors completely regressed including at the injection site. The second patient in this treatment group who had a very advanced stage of esophageal cancer seemed to respond initially but then developed progressive disease. It is not clear which part of the therapy contributed to the responses which could be due to strong inflammatory reactions, control or lack of immunosuppressive factors or a combination of these or other events. Fever was associated in the two groups who received both AT cells and local tumor treatment with either chemotherapy (Protocol III) or radiation (Protocol V), suggesting the release of inflammatory cytokines that are known to cause such reactions. These adverse reactions may, in fact, be required for an immune response to be generated to the tumor.

Chemotherapeutic agents were administered systemically or locally in an attempt to determine if these agents could be used safely and effectively with immunotherapeutic procedures. The doses of chemotherapy were not the amount generally considered optimal for treating the patients' malignancy but rather as support for developing an immune response to the cancer. Chemotherapy may induce apoptosis in the tumor cell augmenting antigen uptake. More importantly, these drugs may effect regulatory T cells. Regulatory cells generally defined as CD4⁺CD25⁺ have been found in most human solid tumors at elevated numbers with frequencies likely correlated to overall survival (Beyer, M. et al., Blood 108: 804-11, 2006). Their elimination by direct targeting of CD25 on the cell surface or preferential destruction is known to result in the generation of tumor-specific and enhanced responses to immunotherapy. The use of low-dose cyclophosphamide, in particular, has been recognized to not only decrease the number of regulatory cells but to also exert immunostimulatory effects. No adverse events were seen when chemotherapy was given systemically prior to DC and AT cell injection. Although the objective of this study was safety and not necessarily therapeutic efficacy, any apparent benefit that was observed by the combination of these modalities must be viewed with the realization that each treatment (chemotherapy, radiation, immature DC and AT cell injection) may have had an effect on the tumor. Indeed, the two patients (head and neck cancer (Protocol V), gastric cancer (Protocol V)) had both failed standard radiation/chemotherapy, but showed substantial responses when receiving a combination of radiation and/or chemotherapy followed by intratumoral injection of DCs and AT cells.

Overall, this study demonstrated that all of the treatment protocols could be relatively well tolerated and that the combination of intratumoral injection of immature DCs and AT cells plus conventional therapy to enhance immunogenicity or override tolerance could be safely and effectively incorporated into single protocols.

While the invention has been particularly shown and described with reference to preferred embodiments thereof, it will be understood by those skilled in the art that various alterations in form and detail may be made therein without departing from the spirit and scope of the invention, as defined by the appended claims.

What is claimed is:

1. A method of reducing the size of tumor cells in tumor tissue of a patient, comprising the steps of:
collecting monocyte cells from a patient;
culturing the monocyte cells with one or a plurality of factors to form immature dendritic cells from the monocyte cells;
introducing the immature dendritic cells and an adjuvant into the tumor tissue of the patient; and
introducing activated T cells into the tumor tissue of the patient.

2. The method according to claim 1, wherein the introduction of the immature dendritic cells, adjuvant and activated T cells is effected by intratumoral injection.

3. The method according to claim 1, wherein said activated T cells are introduced subsequent to introducing the immature dendritic cells and the adjuvant into the patient, in order to generate antigen-specific T cells.

4. The method according to claim 1, wherein said monocyte cells are cultured with GM-CSF and IL-4.

5. The method according to claim 1, wherein said adjuvant is selected from the group consisting of activated lymphocyte medium, super lymphoid tissue extract, β-glucan and keyhole limpet hemocyanin.

6. The method according to claim 5, wherein the activated lymphocyte medium is prepared by using CD3-CD28 beads.

7. The method according to claim 1, wherein activation of the activated T cells is accomplished by exposing T cells to anti-CD3 antibodies and an ionophore.

8. The method according to claim 7, wherein the ionophore is ionomycin.

9. The method according to claim 1, further comprising pretreating the patient with local chemotherapy prior to introducing the immature dendritic cells, adjuvant and activated T cells into the tumor tissue of the patient.

10. The method according to claim 1, further comprising pretreating the patient with systemic chemotherapy prior to introducing the immature dendritic cells, adjuvant and activated T cells into the tumor tissue of the patient.

11. The method according to claim 1, further comprising pretreating the patient with systemic chemotherapy and local tumor irradiation prior to introducing the immature dendritic cells, adjuvant and activated T cells into the tumor tissue of the patient.

12. The method according to claim 1, wherein the patient is a human.

13. A cancer vaccine which when introduced into tumor tissue combines with an antigen for the reduction of the size of a tumor in a patient, said cancer vaccine comprising immature dendritic cells derived from monocyte cells collected from the patient, an adjuvant, antigens from the tumor in the patient, and activated T cells.

14. The cancer vaccine according to claim 13, wherein said vaccine is introduced into the tumor tissue of the patient by intratumoral injection.

15. The cancer vaccine according to claim 13, wherein said monocyte cells are cultured with GM-CSF and IL-4.

16. The cancer vaccine according to claim 13, wherein said adjuvant is selected from the group consisting of activated lymphocyte medium, super lymphoid tissue extract, β-glucan and keyhole limpet hemocyanin.

17. The cancer vaccine according to claim 16, wherein the activated lymphocyte medium is prepared by using CD3-CD28 beads.

18. The cancer vaccine according to claim 13, wherein activation of the activated T cells is effected by exposing T cells to anti-CD3 antibodies and an ionophore.

19. The cancer vaccine according to claim 18, wherein the ionophore is ionomycin.

20. The cancer vaccine according to claim 13, wherein said patient is a human.
